(19) United States
(12) Patent Application Publication
(10) Pub. No.: US 2011/0054375 A1
(43) Pub. Date: Mar. 3, 2011

(54) Dressing comprising polyacrylate particles and use thereof

(76) Inventor: Hans Smola, Koln (DE)

(21) Appl. No.: 12/744,681

(22) PCT Filed: Nov. 26, 2007
(86) PCT No.: PCT/EP08/09998

§ 371 Date: Oct. 19, 2010

(30) Foreign Application Priority Data
Nov. 27, 2007 (EP) 07022915.8

(51) Int. Cl. A61L 15/22; A61K 9/14; A61K 31/765; A61P 17/02; A61F 13/00

(52) U.S. Cl. 602/48; 424/501; 424/78.06

(57) ABSTRACT

The invention relates to dressings comprising a particle mixture of polyacrylate particles for using in modern wound treatment. The invention especially relates to a dressing comprising a particle mixture of polyacrylate particles, which contains a particle fraction for inhibiting proteases in a wound and a particle fraction for absorbing or eliminating aqueous liquids.

The invention concerns dressings comprising a particle mixture of polyacrylate particles for use in modern wound treatment.

The healing of skin wounds is based on the ability of the skin to regenerate epithelia as well as connective and supporting tissue. It is characterized as a complex occurrence of interrelated cell activities, which gradually advance the healing process. Literature describes three essential healing phases of a wound independently of the type of wound. This includes the inflammatory or exudative phase for haemostasis and cleaning of the wound (phase 1, cleaning phase), the proliferative phase for building up granulation tissue (phase 2, granulation phase) and the differentiation phase for epithelization and scar formation (phase 3, epithelization phase).

Literature describes numerous proposals for supporting the individual wound healing phases. In particular, dressings with polyacrylate particles have been the subject matter of numerous articles in technical literature as well as patent specifications for some time. The US patent specification U.S. Pat. No. 5,977,428 A for example describes an absorbing dressing which comprises dried absorbing hydrogel particles within a porous cover. These hydrogel particles may consist of polyacrylate, are used in the form of a granulate or powder and are present within a pocket forming the dressing, of a textile material or a nonwoven, possibly mixed with a binding agent. US patent specification U.S. Pat. No. 7,230,154 B2 moreover describes a foamed dressing which has an adhesive wound contact layer of silicon. Polyacrylate particles are introduced into the foamed element, which may have a defined grain size of between 100 and 900 μm. The European patent application EP 1688109 A1 describes a dressing for treating trophic disturbances in the wound. Carbon dioxide is thereby used as a treating agent. A depot for an aqueous phase is introduced into the dressing, which contains polyacrylate particles having a defined particle distribution, wherein the portion of particles having a particle size of between 850 and 300 μm should amount, in particular, to more than 70 weight % with respect to the overall weight of the particles. These documents have in common that polyacrylate particles are always used as a storage medium or absorbing medium for aqueous liquids.

It is therefore the underlying purpose of the present invention to provide an improved means for the treatment of wounds. Moreover, a dressing shall be presented which influences the pathological state of a wound in such a fashion that a normal natural wound healing process takes place.

This object is achieved by a dressing in accordance with claim 1. In accordance thereto, an inventive dressing comprises a particle mixture and a carrier material for the particle mixture for inhibiting proteases in a wound and/or for hydroactive wound treatment, wherein the dressing comprises at least 10 weight % of polyacrylate particles (with respect to the overall amount of carrier material) and wherein the particle mixture contains

a) 5 to 100 weight % of particles having a particle size x with x≦300 μm as a means for inhibiting proteases in the wound, and
b) 0 to 95 weight % particles having a particle size x with x>300 μm as a means for absorbing and/or eliminating aqueous solutions.

The inventive dressing thereby preferably comprises a particle mixture of polyacrylate particles which contains

a) 5 to 98 weight % of particles having a particle size x with x≦300 μm as a means for inhibiting proteases in the wound; and
b) 2 to 95 weight % of particles having a particle size x with x>300 μm as a means for absorbing and/or eliminating aqueous solutions.

With particular preference, it is thereby provided that the inventive dressing comprises a particle mixture of polyacrylate particles which contains

a) 20 to 80 weight % of particles having a particle size x with x≦300 μm as a means for inhibiting proteases in the wound; and

b) 20 to 80 weight % of particles having a particle size x with x>300 μm as a means for absorbing and/or eliminating aqueous solutions.

An inventive dressing therefore comprises a particle mixture with a defined amount of a first particle fraction having a defined particle size and a second amount of a second particle fraction having a second particle size that differs from the first particle size, wherein each particle fraction contains polyacrylate particles. The polyacrylate particles of one size range may thereby be composed of particles having the same or a different size, wherein the particle size(s) is/are within the range. In connection with the present invention, the amount of particles, in particular of one single size range, is always stated in weight % with respect to the overall amount of particles, unless otherwise stated.

The particle mixture may additionally also contain polyacrylate particles consisting of different polyacrylates, i.e. the particle mixture comprises at least two different polyacrylate particle types. The polyacrylate particles of the first size range a) may, in particular, also differ from the polyacrylate particles of the second size range b). The polyacrylates may e.g. differ with respect to the neutralization degree, linking degree, linking agent and/or the copolymers. In the simplest case, it is possible to also use polyacrylate particles which are identical with respect to their structure and only differ with respect to the stated particles sizes. The particles of the first or second particle size range may, however, also consist of different polyacrylate particles, i.e. the particles of the first size range contain first polyacrylate particles and second polyacrylate particles which are different from the first ones, wherein both types comprise polyacrylate particles which are within the respective size range.

The present invention thereby defines a particle mixture as a mixture, the individual components (particles) of which may be present either spatially next to each other, partially mixed through, completely mixed through or spatially separated from each other, wherein the mixture is to be regarded in any case as a component of a dressing. The particles of the individual particle size ranges may, in particular, also be spatially next to each other, partially mixed through, completely mixed through or spatially separated from each other.

In connection with the present invention, the particle size is determined analogously to EDANA 420.2-02, wherein the screens (diameter 200 mm) have hole sizes that correspond to the specifications. It is moreover also possible to use screens having different hole sizes such as e.g. 125 μm, 160 μm, 630 μm, 900 μm and 1500 μm. This is based on dry polyacrylate particles having a moisture content of less than 10 weight % of water with respect to the overall weight of the particles, wherein the moisture content is determined in accordance with EDANA 450.2-02.

It has surprisingly turned out that polyacrylate particles inhibit proteases via diffusible mechanisms and also compartment them through direct attachment, thereby withdrawing them from the wound exudate or the wound. In particular, it has turned out that polyacrylate particles are suited to inhibit proteases in chronic wounds. It has also turned out that metallo proteases are bound or compartmented by polyacrylate particles such that these metallo proteases can be removed with the polyacrylate particles from wound fluid or a wound. In this fashion, an excess of metallo proteases in chronic wounds can be absorbed by means of the polyacrylate particles such that a natural healing process can take place. Surprisingly, polyacrylate particles having a particle size x with x≦300 μm are particularly suited in this case. Particles of this size inhibit the activity of proteases which prevent wound healing, in particular metallo proteases, in a wound with particular efficiency, wherein particles of this size have an at least four times as large an affinity for metallo proteases compared to larger particles. For this reason, polyacrylate particles having a particle size x with x≦300 μm are particularly well suited for the treatment of chronic wounds. Normal dressings with polyacrylate particles for the treatment of chronic wounds contain polyacrylate particles having a particle size x with x>300 μm, since these particles are in most cases well capable of absorbing wound exudate or are used as storage medium for aqueous liquids. Polyacrylate particles having a particle size x with x≦300 μm are less suitable or unsuitable for absorbing wound exudate, since these particles can absorb or hold a much smaller amount of aqueous liquids compared to particles having a particle size x with x>300 μm. For this reason, the present dressing provides a wound treating means due to the specific selection of a particle mixture having particles of defined sizes, which inactivates proteases, which prevent healing, in a wound and also provides hydroactive properties.

An excess of, in particular, metallo proteases is disturbing, in particular, in the granulation phase of wound healing, since the disturbed balance between metallo proteases and newly built-up connective tissue prevents sufficient tissue build-up. Tissue build-up thereby critically depends on the stabilization of newly synthesized extra-cellular matrix molecules, which are assembled to form a three-dimensional tissue, which is simultaneously and subsequently accompanied by angiogenesis and proliferation of cells into the wound area.

It has turned out that polyacrylate particles having a particle size of x≦300 μm can be used in the granulation phase, in particular, for badly healing wounds. Any potential inflammation has disappeared or is non-existent in this phase, such that granulation and thereby cell growth can take place as long as there is no metal protease excess. This excess can be eliminated with great efficiently by means of polyacrylate particles of the stated size. In this fashion, tissue build-up is promoted by eliminating the mechanisms that disturb wound healing, which clinically shows in the form of an improvement of the state of the wound and of a reduction in size of the wound in that the wound volume and/or the wound size decreases. For this reason, the use of polyacrylate particles having a particle size x with x≦300 μm for blocking or inhibiting proteases, in particular, serine proteases or metallo proteases for tissue build-up and/or for regulating the tissue build-up in chronic wounds is also subject matter of the present invention.

In connection with the present invention, proteases (peptidases, peptide hydrolases) are defined as enzymes which catalyze the hydrolytical decomposition of a peptide compound into proteins and peptides (proteolysis). Proteases therefore systematically belong to the group of hydrolases. Proteases are classified with respect to the location of decomposition in the substrate. Thus, the decomposition of peptide compounds inside peptides or proteins is catalyzed by endopeptidases (proteinases), whereas peptide compounds are decomposed at the end of a peptide or protein molecule by exopeptidases (former peptidases).

The proteases can thereby be further differentiated with respect to their groups which are responsible for the catalysis in the active center. One differentiates e.g. a) serine proteases, b) cystein proteases, c) aspartate proteases and d) metallo proteases. The group of metallo proteases (metallo peptidases) have e.g. metal ions in their active center which participate in the catalytic mechanism of proteolysis. These metal ions, in particular, divalent metal cations such as magnesium, zinc, calcium, iron i.a. are also regarded as coenzyme. In accordance with the above-described distinguishing feature, there are furthermore a) metallo endopeptidases (metallo proteinases) and b) metallo exopeptidases.

Matrix metallo proteases (MMP), also called matrixines, belong to a family of proteases which are defined by structural homologies. Their enzymatic activity depends on metal ions in the active center. Metallo proteases were initially identified by their role in tissue restructuring, in particular, by the decomposition of the extra-cellular matrix. This group of proteases systematically belongs to the metallo endopeptidases (metallo proteinases). The matrix metallo proteinases include i.a. collagenases, gelatinases, stromelysines, matrilysines. An overview and classification are given in technical literature in Parks, Matrix Metalloproteinases, Biology of Extracellular Matrix Series, Ed. Mecham, R. P., Academic Press, Inc., San Diego, Calif. (1998) and in table 1 listed below. Table 1 shows synonyms and, if necessary, the enumeration of the enzymes in accordance with the Enzym Commision (EC). The matrix metallo proteinases are synthesized and secreted as inactive precursors. They are transferred into the active form by complex mechanisms which are currently not finally clarified in detail. All metallo proteases can be inhibited by ethylene diamine tetra acetic acid (EDTA).

TABLE 1
Matrix metallo proteases (MMP)

| Enzyme | E.C. no. | pseudonym |
|---|---|---|
| MMP-1 | 3.4.24.7 | Collagenase-1, fibroblastic |
| | | collagenase |
| MMP-8 | 3.4.24.34 | Collagenase-2, neutrophilic |
| | | collagenase |
| MMP-13 | | Collagenase-3, |
| MMP-18 | | Collagenase-4 |
| MMP-2 | 3.4.24.24 | Gelatinase-A |
| MMP-9 | 3.4.24.35 | Gelatinase-B |
| MMP-3 | 3.4.24.17 | Stromelysine-1, transine-1, |
| | | procollagenase |
| MMP-10 | 3.4.24.22 | Stromelysine-2; transine-2 |
| MMP-11 | | Stromelysine-3 |
| MMP-7 | 3.4.24.33 | Matrilysine, PUMP-1 |
| | | protease |
| MMP-29 | | Matrilysine-2, endometase |
| MMP-20 | | Enamelysine |
| MMP-28 | | Epilysine |
| MMP-12 | 3.4.24.65 | Macrophagic metallo |
| | | elastase |
| MMP-19 | | RASI-1 |
| MMP-23 | | CA-MMP |
| MT1-MMP | 3.4.24.80 | Membrane-type MMP-14 |
| MT2-MMP | | Membrane-type MMP-15 |
| MT3-MMP | | Membrane-type MMP-16 |
| MT4-MMP | | Membrane-type MMP-17 |
| MT5-MMP | | MMP-24 |
| MT6-MMP | | MMP-25, leucolysine |

Serine-proteases have an L-serine residue in the active center, which is essential for catalysis and can be inhibited by diisopropyl fluorophosphates. A decisive function in wound healing is also attributed to these proteases. The group of serine-proteases contains e.g. chromotrypsine, elastase, kallikrein, plasmine, trypsine, thrombine, etc. Most conventional serine proteases have residues of the amino acids L-histidine and L-asparagine in their active center in addition to the L-serine residue. All three amino acid residues participate in a cascade of reactions during protolysis, in which a proton of the L-serine residue is transferred to the substrate. When the proteinases have a serine-dependent mechanism, they are also called serine-proteinases. The effect of these serine proteinases on wound healing is also inhibited by polyacrylate particles having a particle size x with x≦300 μm.

Accordingly, an inventive dressing as a means for inhibiting proteases in a wound comprises a particle mixture and a carrier material for the particle mixture, wherein the dressing comprises at least 10 weight % of polyacrylate particles (with respect to the overall amount of carrier material) and wherein the particle mixture contains

a) 5 to 100 weight % of particles having a particle size x with x≦300 μm as a means for inhibiting proteases in the wound; and
b) 0 to 95 weight % of particles having a particle size x with x>300 μm as means for absorbing and/or eliminating aqueous solutions.

An aqueous solution in this context and in connection with the present invention means water, salt solutions, in particular, physiological salt solutions such as physiological sodium chloride solutions or Ringer solutions and wound exudate.

In accordance with a second embodiment of the inventive dressing, the dressing comprises a particle mixture containing a first particle fraction a) with a limited size range as a means for inhibiting proteases in the wound and a second particle fraction b) as a means for absorbing and/or eliminating aqueous solutions. The dressing thereby comprises, in particular, a particle mixture which contains

a) 5 to 100 weight % of particles having a particle size x with x≦300 μm as a means for inhibiting proteases in the wound, and
0 to 5 weight % of particles having a particle size x with x≦45 μm.

The particle mixture thereby contains, in particular

a) 5 to 100 weight % of particles having a particle size x with x≦300 μm as a means for inhibiting proteases in the wound, and
0 to 4 weight %, in particular 0 to 3 weight %, in particular 0 to 2 weight %, in particular 0 to 1 weight %, and preferentially 0 weight % of particles having a particle size x with x≦45 μm.

The dressing moreover comprises, in particular, a particle mixture which contains

a) 5 to 100 weight % of particles having a particle size x with x≦300 μm as a means for inhibiting proteases in the wound, and
0 to 5 weight % of particles having a particle size x with x≦150 μm.

The particle mixture thereby preferably contains

a) 5 to 100 weight % of particles having a particle size x with x≦300 μm as a means for inhibiting proteases in the wound, and
0 to 4 weight %, in particular 0 to 3 weight %, in particular 0 to 2 weight %, in particular 0 to 1 weight %, and preferentially 0 weight % of particles having a particle size x with x≦150 μm.

An inventive dressing therefore comprises with particular preference a particle mixture of polyacrylate particles which contains

a) 5 to 100 weight % of particles having a particle size x with 150<x≦300 μm as a means for inhibiting proteases in the wound, and
b) 0 to 95 weight % of particles having a particle size x with x>300 μm as means for absorbing and/or eliminating aqueous solutions.

When only particles having a particle size x with 150<x≦300 μm are used as means for inhibiting proteases in the wound, a dressing is provided which removes with particular efficiency proteases, in particular, metallo proteases from a wound, which are detrimental to healing. On the other hand, a particularly safe dressing is provided. Particles having a particle size x with x≦150 μm and, in particular, particles having a particle size x with x≦45 μm easily gain access into a wound when no particular barriers are provided, which is, however, not desired.

In accordance with a further embodiment of the inventive dressing, the dressing comprises a particle mixture which contains a first particle fraction a) as a means for inhibiting proteases in the wound and a second particle fraction b) with a limited size range as a means for absorbing and/or eliminating aqueous solutions. In this connection, the dressing preferably comprises a particle mixture which contains

a) 5 to 100 weight % of particles having a particle size x with x≦300 μm as a means for inhibiting proteases in the wound, and
b) 0 to 95 weight % of particles having a particle size x with 300<x≦1500 μm as means for absorbing and/or eliminating aqueous solutions.

The dressing moreover preferably comprises a particle mixture which contains

a) 5 to 100 weight % of particles having a particle size x with 45<x≦300 μm as a means for inhibiting proteases in the wound, and
b) 0 to 95 weight % of particles having a particle size x with 300<x≦1500 μm as a means for absorbing and/or eliminating aqueous solutions.

It has turned out that a dressing comprising a particle mixture of polyacrylate particles having particles of a particle size x with 300<x≦1500 μm has excellent absorption and retaining properties as means for absorbing and/or eliminating aqueous solutions.

In this connection, 0 to 90 weight %, in particular 0 to 80 weight %, in particular 0 to 60 weight %, in particular 0 to 40 weight %, in particular 0 to 20 weight % and preferentially, in particular 0 to 10 weight % are thereby used as a means for absorbing and/or eliminating aqueous solutions. It may, however, also be the case that no particles having a particle size x with 300<x≦1500 μm are used as a means for absorbing and/or eliminating aqueous solutions.

An inventive dressing may alternatively also comprise a particle mixture which contains a first particle fraction a) as a means for inhibiting proteases in the wound, and a second particle fraction b) with a further limited size range as a means for absorbing and/or eliminating aqueous solutions. In this connection, the dressing comprises, in particular, a particle mixture of polyacrylate particles which contains

b) 0 to 95 weight % of particles having a particle size x with 300<x≦1500 μm as a means for absorbing and/or eliminating aqueous solutions, and
0 to 5 weight % of particles having a particle size x with x>850 μm.

The particle mixture thereby contains, in particular

b) 0 to 95 weight % of particles having a particle size x with 300<x≦1500 as a means for absorbing and/or eliminating aqueous solutions and
0 to 4 weight %, in particular 0 to 3 weight %, in particular 0 to 2 weight %, in particular 0 to 1 weight %, and preferentially 0 weight % of particles having a particle size x with x>850 μm.

An inventive dressing may alternatively also comprise a particle mixture which contains a first particle fraction a) as a means for inhibiting proteases in the wound and a second particle fraction b) with a further limited size range as a means for absorbing and/or eliminating aqueous solutions. The dressing thereby comprises, in particular, a particle mixture of polyacrylate particles which contains

b) 0 to 95 weight % of particles having a particle size x with 600<x≦850 μm as a means for absorbing and/or eliminating aqueous solutions, and
0 to 5 weight % of particles having a particle size x with 300<x≦600 μm.

The particle mixture thereby contains, in particular,

b) 0 to 95 weight % of particles having a particle size x with 600<x≦850 μm as a means for absorbing and/or eliminating aqueous solutions, and
0 to 4 weight %, in particular 0 to 3 weight %, in particular 0 to 2 weight %, in particular 0 to 1 weight % and preferentially 0 weight % of particles having a particle size x with 300<x≦600 μm.

In accordance with a further development of the invention, a dressing is preferred which comprises a particle mixture of polyacrylate particles containing

a) 5 to 100 weight % of particles having a particle size x with 150<x≦300 μm as a means for inhibiting proteases in the wound; and
b) 0 to 95 weight % of particles having a particle size x with 600<x≦850 μm as a means for absorbing and/or eliminating aqueous solutions.

This dressing comprises, in particular, a particle mixture of polyacrylate particles containing

a) 5 to 98 weight % of particles having a particle size x with 150<x≦300 μm as a means for inhibiting proteases in the wound; and
b) 2 to 95 weight % of particles having a particle size x with 600<x≦850 μm as a means for absorbing and/or eliminating aqueous solutions.

With particular preference, this dressing comprises a particle mixture of polyacrylate particles which contains

a) 20 to 80 weight % of particles having a particle size x with 150<x≦300 μm as a means for inhibiting proteases in the wound, and
b) 20 to 80 weight % of particles having a particle size x with 600<x≦850 μm as a means for absorbing and/or eliminating aqueous solutions.

Alternatively, however, the dressing may comprise a particle mixture of polyacrylate particles, which contains

a) 5 to 100 weight % of particles having a particle size x with 150<x≦300 μm as a means for inhibiting proteases in the wound, and
b) 0 to 95 weight % of particles having a particle size x with 850<x≦1500 μm as a means for absorbing and/or eliminating aqueous solutions.

The used polyacrylate particles having a particle size x with 850<x≦1500 μm show a particularly good performance with respect to their absorption capacity and/or eliminating capacity for aqueous solutions, in particular, for carrier materials comprising a material which differs from fiber material, e.g. a foamed material. The selection of the size ranges of the particles for absorbing and/or eliminating aqueous solutions must therefore be adjusted to the respective carrier material.

It has turned out that the particle mixture of polyacrylate particles having a particle size x with x≦300 μm, in particular with 45<x≦300 μm and preferentially 150<x≦300 μm, which is an essential component of the dressing, binds or compartments proteases, in particular metallo proteases such as e.g. matrix metallo proteases MMP-2 or MMP-9 very effectively. This is advantageous in that the dressing reduces an excess amount of proteases, in particular metallo proteases, to a reduced amount that is required for natural wound healing. These bound metallo proteases are no longer available as inhibitors of wound healing in the wound area. These proteases can moreover be effectively removed by changing the dressing. An inventive dressing therefore comprises a particle mixture of polyacrylate particles having a particle size x with x≦300 μm as protease inhibitor, wherein the proteases are bound and/or compartmented by the polyacrylate particles.

When a specific dressing is to be provided to support severely disturbed wound healing with a high concentration of inhibitors of wound healing, it has turned out that a dressing is particularly preferred which comprises a particle mixture of polyacrylate particles with

a) 20 to 100 weight % of particles having a particle size x with x≦300 μm, in particular with 45<x≦300 μm, and preferentially 150<x≦300 μm as a means for inhibiting proteases in the wound, and
b) 0 to 80 weight % of particles having a particle size x with x>300 μm as a means for absorbing and/or eliminating aqueous solutions.

One dressing was thereby particularly preferred, which comprises a particle mixture of polyacrylate particles with

a) 50 to 100 weight % of particles having a particle size x with x≦300 μm, in particular with 45<x≦300 μm, and preferentially 150<x≦300 μm as a means for inhibiting proteases in the wound, and
b) 0 to 50 weight % of particles having a particle size x with x>300 μm as a means for absorbing and/or eliminating aqueous solutions.

If otherwise a dressing is to be provided which shall support only slightly disturbed wound healing with a low concentration of inhibitors of wound healing, it has turned out that a dressing is particularly preferred, which comprises a particle mixture of polyacrylate particles with

a) 5 to 50 weight % of particles having a particle size x with x≦300 μm, in particular with 45<x≦300 μm, and preferentially 150<x≦300 μm as a means for inhibiting proteases in the wound, and
b) 50 to 95 weight % of particles having a particle size x with x>300 μm as a means for absorbing and/or eliminating aqueous solutions.

These compositions can be adjusted to the requirements of a wound. They can increasingly bind inhibitors of wound healing or, if these are not present in markably high concentrations, interfere as little as possible with the normal wound healing by intercepting growth factors and other endogenous mediators which are physiologically regulated in normal wound healing.

Within the scope of the present invention, the term polyacrylate particles is defined by those particles which are formed from a polyacrylate. A polyacrylate of this type is a synthetic polymer comprising as monomer (M1) acrylic acid (2-propenoic acid, CH₂═CH—CO₂H) and/or a salt thereof having a monomer portion of more then 70 weight % of acrylic acid and/or a salt thereof (with respect to the overall weight of the polyacrylate). Inventive polyacrylates have, in particular, a monomer portion of more than 80 weight % of acrylic acid and/or a salt thereof and with particular preference more than 95 weight % of acrylic acid and/or a salt thereof with respect to the overall weight of the polyacrylate. An inventive dressing therefore comprises, in particular, a particle mixture of polyacrylate particles having a monomer portion of more than 80 weight % of acrylic acid and/or a salt thereof and, with particular preference, more than 95 weight % of acrylic acid and/or a salt thereof with respect to the overall weight of the polyacrylate, in particular, as a means for inhibiting proteases in a wound. The polyacrylate may thereby be present in the form of a homopolymer, copolymer or block polymer. When the polyacrylate is present in the form of a copolymer or block polymer, the monomer portion of the monomer M1 in the polymer is in any case more than 70%, in particular, more than 80%, and preferentially more than 95% with respect to the overall weight of the polyacrylate. These copolymeric polyacrylates or block-polymeric polyacrylates may contain, in addition to the monomer M1, as comonomers M2, in particular α,β-unsaturated ethers (vinyl ether), α,β-unsaturated carboxylic acids or α,β-unsaturated carboxylic acid ester, (vinyl ester). Particularly preferred among the comonomers M2 of the α,β-unsaturated carboxylic acids are methacrylic acid (2-methyl propenoic acid), ethacrylic acid (2-ethyl propenoic acid), crotonic acid (2-butenoic acid), sorbic acid (trans-trans-2,4 hexadienenoic acid, maleic acid (cis-2-butenedioic acid) or fumaric acid (trans-2-butendioic acid). In one particularly preferred form of the invention, the polyacrylate may also consist of a) a homopolymer of acrylic acid and/or b) a copolymer of i) acrylic acid and a salt of the acrylic acid, ii) of methacrylic acid and a salt of methacrylic acid or iii) of acrylic acid and methacrylic acid and the salts thereof. The polyacrylate may furthermore also be a mixture of different polyacrylates.

In this connection, in particular, the α,β-unsaturated carboxylic acids as well as the acrylic acids may be present in the neutralized form as salt, in non-neutralized form as free acid or mixtures thereof. Polyacrylates which are formed from acrylic acid and salts of the acrylic acid have turned out to be particularly effective. In particular, alkaline metal or alkaline earth metal salts must be particularly emphasized in this connection. In particular, polyacrylates which consist of homopolymers and/or copolymers, which, as monomers, comprise acrylic acid and/or sodium acrylate or potassium acrylate, have turned out to be particularly effective.

An inventive dressing preferably comprises moreover a particle mixture of polyacrylate particles which consist of homopolymers and/or copolymers which comprise as monomers acrylic acid and/or sodium acrylate or potassium acrylate, in particular as a means for inhibiting proteases.

It has moreover surprisingly turned out that the present object is achieved in an outstanding fashion by polyacrylates from the group of the linked and/or crosslinked and/or surface-linked polyacrylates. These polyacrylates comprise preferably a) a homopolymer which consists of the monomers M1 and is linked and/or crosslinked by means of a crosslinker, and/or b) a copolymer which consists of the monomers M1 and M3, wherein the monomer M1 is acrylic acid and/or a salt thereof, and the monomer M3 is selected from the group of the crosslinkers. This means that these polyacrylates comprise a polyacrylate and/or a polyacrylate which is copolymerized from acrylic acid and/or a salt thereof and a crosslinker, which are subsequently linked by means of a crosslinker.

An inventive dressing moreover preferably comprises a particle mixture of polyacrylate particles which comprise a linked and/or crosslinked and/or surface-linked polyacrylate, in particular as a means for inhibiting proteases.

In particular, it has turned out that linked and/or crosslinked polyacrylates are particularly effective, which contain compounds V1 as crosslinker, which have at least two ethylenically unsaturated groups within one molecule, or compounds V2 which have at least two functional groups which can react with functional groups of acrylic acid and/or a salt thereof in a condensation reaction, in an addition reaction or in a ring opening reaction, or compounds V3 which have at least one ethylenically unsaturated group and at least one functional group which can react with functional groups of the acrylic acid and/or a salt thereof and/or the α,β-unsaturated comonomers in a condensation reaction, in an addition reaction or in a ring opening reaction. The compounds V1 thereby yield linking of the polymers through radical polymerization of the ethylenically unsaturated groups of the linker molecule with the monethylenically unsaturated monomers acrylic acid and/or a salt thereof and/or one of the α,β-unsaturated comonomers, whereas compounds V2 yield linking of the polymers through condensation reaction of the functional groups with the functional groups of the acrylic acid and/or one of its salts or a α,β-unsaturated of the comonomers. With respect to compounds V3, the polymer is correspondingly linked both through radical polymerization of the ethylenically unsaturated group and also through condensation reaction between the functional group of the linker and the functional groups of the monomers.

Preferred compounds V1 are polyacrylates or polymethacrylates which are obtained e.g. by converting a polyol such as e.g. ethylene glycol (1,2-ethanediol), propylene glycol (1,2-propandiol), trimethylol propane (2-ethyl-2-hydroxymethyl-1,3-propandiol), 1,6-hexandiol, glycerine (1,2,3-propantriol), pentaerythrit (2,2-bis(hydroxymethyl)propane-1,3-diol), polyethylene glycol (HO—(CH₂—CH₂—O)ₙ—H with n=2 to 20) with n=1 to 20), polypropylene glycol (HO—(CH)CH₃)—CH₂—O)ₙ—H with n=2 to 20), an aminoalcohol, a polyalkylene polyaminens such as e.g. diethylenetriamine or triethylenetriamine or an alcoxylated polyol with acrylic acid or methacrylic acid. With particular preference, the linked polyacrylate is a polyacrylate which is linked by means of a compound V1 which is a di-, tri- or tetraester of the polyacrylate or polymethacrylate and is obtained by converting an alcoxylated polyol, in particular an ethoxylated polyol, in particular, ethoxylated ethylene glycol, ethoxylated propylene glycol, ethoxylated trimethylol propane, ethoxylated 1,6-hexandiol or ethoxylated glycerine with an average number of ethylene oxide units n per hydroxy group of n=1 to 10 with acrylic acid or methacrylic acid. Preferred compounds V1 are moreover polyvinyl compounds, polyallyl compounds, polymethylallyl compounds, acrylic acid esters or methacrylic acid esters of a monovinyl compound, acrylic acid ester or methacrylic acid ester of a monoallyl compound or monomethyl allyl compound, preferably of monoallyl compounds or monomethylallyl compounds of a polyol or an amino alcohol. In this connection, reference is made to DE 195 43 366, DE 195 43 368.

In any case, the polyacrylate particles from the above-mentioned polyacrylates have the above-mentioned particle sizes in the dressing in a dry state. These particles can be used in the dressing in a dry or already soaked state. In the soaked state, the polyacrylate particles are present in the form of gel-shaped particles. Water, physiological sodium chloride solution or Ringer solution may thereby be added to the polyacrylate particles.

An inventive dressing has, in particular, a particle mixture of polyacrylate particles, which comprises at most 10 weight % of water (with respect to the overall weight of the polyacrylate particles) prior to use, i.e. the polyacrylate particles have a moisture content of at most 10%. The dressing thereby comprises, in particular, a particle mixture which comprises at most 7 weight %, preferably at most 5 weight % and preferentially at most 4 weight % of water (with respect to the overall weight of the polyacrylate particles).

In accordance with a progressing idea, an inventive dressing may be provided in the form of a multilayer dressing. In this connection, a second further layer of the dressing is used in the dressing in addition to a first layer comprising the particle mixture and the carrier material for the particle mixture. This second layer can assume diverse functions. In this connection, the dressing comprises moreover, in particular, a wound contact layer separating the particle mixture and the carrier material for the particle mixture from a wound, or furthermore a cover surrounding the particle mixture and the carrier material for the particle mixture. This ensures, in particular, that in the state of intended use of the dressing, no particles get into the wound. This dressing comprising the particle mixture, the carrier material and the cover therefore comprises a first layer comprising the particle mixture and the carrier material, and a second and third layer formed by the cover. The cover or the wound contact layer can thereby be formed, in particular, from a nonwoven, a knitted fabric, a fabric or a woven fabric, wherein this wound contact layer or this cover preferably furthermore comprises no particles. Nonwovens are thereby particularly preferred, since these materials are very dense and no particles can get into a wound.

For this reason, a dressing which is formed as a multilayer dressing and comprises, in addition to the layer comprising the particle mixture and the carrier material for the particle mixture, a second layer as a wound contact layer, is also a subject matter of the present invention, wherein this wound contact layer contains, in particular, no particles.

In particular, dressings which do not stick to the wound are also subject matter of the present invention. These dressings support to a particular degree wound healing in the granulation phase of wound healing, since they inhibit the activity of proteases due to the content of particles with a particle size x with x≦300 μm, in particular through compartmentation or bonding and do not damage newly grown tissue when the dressing is changed. The wound contact layer of these dressings which do not stick to the wound is, in particular, a knitted fabric, a fabric, a woven fabric or a non-woven fabric which does not stick to the wound and moreover preferably consists of a hydrophobic fiber material. The wound contact layer may, in particular, be a knitted fabric, a fabric or a woven fabric of a hydrophobic polyethylene, polypropylene, polyester or viscose polymer material. Due to the design in the form of a knitted fabric, a fabric or a woven fabric, the wound contact layer can be stretched or deformed in one or more directions and does not automatically contract or realign itself again. The surface of such a wound contact layer moreover adapts with exact fit to the surface of the skin or wound to be treated.

The dressing may alternatively also comprise a particle mixture of polyacrylate particles and a carrier material for the particle mixture, wherein the carrier material comprises a hydrophilic fiber material and wherein, in particular, the polyacrylate particles comprise a linked and/or crosslinked polyacrylate. In particular, water-insoluble fibers of cellulose, in particular, largely delignified technical cellulose fibers, in particular, wood pulp fibers, in particular of a fiber length of <5 mm can thereby be used as hydrophilic fiber material. The fiber material may also contain hydrophilic fiber material of regenerated cellulose, carboxymethyl cellulose, carboxyethyl cellulose, hydroxymethyl cellulose or hydroxyetyhl cellulose. Alternatively a fiber mixture of cellulose, regenerated cellulose, carboxymethyl cellulose, carboxyethyl cellulose, hydroxymethyl cellulose or hydroxy ethyl cellulose fibers and fibers of polyethylene, polypropylene or polyester may be provided. In one particularly preferred embodiment, the dressing comprises a particle mixture of polyacrylate particles of the stated composition and a mixture of cellulose fibers, polypropylene fibers as carrier material for the particle mixture.

Another subject matter of the present invention is therefore a dressing comprising a particle mixture of the above-mentioned composition and a carrier material for the particle mixture which contains as first fibers staple fibers of synthetic and/or natural polymers. This dressing comprises, in particular, first hydrophilic staple fibers. These fibers may be processed in a so-called air-laid process together with the particle mixture to form a layer.

Depending on the intended use, an inventive dressing may comprise different amounts of polyacrylate particles and carrier material for the polyacrylate particles. In accordance with the invention, the dressing comprises at least 10 weight % of polyacrylate particles (with respect to the carrier material), wherein the polyacrylate particles have the composition stated in connection with the present invention. However, dressings having at least 20 weight %, in particular at least 25 weight % and preferentially at least 30 weight % of polyacrylate particles (with respect to the carrier material) are particularly preferred. In order not to limit the performance characteristics of the dressing with respect to inhibiting the activities of proteases and/or absorption and/or elimination of aqueous liquids, it should be ensured that the content of polyacrylate particles with respect to the carrier material is, in particular, not more than 80 weight % and in particular not more than 75 weight %.

In another alternative embodiment, an inventive multilayer dressing comprises a first particle-containing layer A and at least one second particle containing layer B. The first layer A thereby comprises particles of a first size and the second layer B comprises particles of a second size which differs from the first size.

For this reason, another dressing presents a preferred embodiment of the present invention, which comprises a particle mixture and a carrier material for the particle mixture, wherein the particle mixture contains polyacrylate particles of different sizes, and wherein the dressing comprises a first particle-containing layer A and at least one second particle-containing layer B, and wherein the first layer A comprises

a) 5 to 98 weight % of particles having a particle size x with x≦300 μm, in particular with 45<x≦300 μm, and preferentially 150<x≦300 μm as a means for inhibiting proteases in the wound;
and the second particle-containing layer B comprises
b) 2 to 95 weight % of particles having a particle size x with x>300 μm, in particular 300<x≦1500 μm, in particular 300<x≦850 μm, and preferentially 600<x≦850 μm as a means for absorbing and/or eliminating aqueous solutions, with the requirement that the content of particles is supplemented up to 100% and that the overall content of particles with respect to the carrier material for the particles is at least 10 weight %.

The first layer A thereby comprises, in particular

a) 5 to 95 weight %, in particular 20 to 80 weight % and preferentially 50 to 80 weight % of polyacrylate particles having a particle size x with x≦300 μm, in particular with 45<x≦300 μm, and preferentially with 150<x≦300 μm as a means for inhibiting proteases in the wound; and the second particle-containing layer B comprises
b) 5 to 95 weight %, in particular 20 to 80 weight % and preferentially 20 to 50 weight % of polyacrylate particles having a particle size x with x>300 μm, in particular 300<x≦1500 μm, in particular 300<x≦850 μm, and preferentially 600<x≦850 μm as a means for absorbing and/or eliminating aqueous solutions.

Depending on the intended use, a multilayer dressing may also comprise different amounts of polyacrylate particles and carrier material for the polyacrylate particles. When the particles are to be arranged in different layers, the particle amount should be at least 10 weight % with respect to the carrier material of all particle-containing layers. In particular, dressings having at least 20 weight %, in particular at least 25 weight % and preferentially at least 30 weight % of polyacrylate particles (with respect to the carrier material of all particle-containing layers) are particularly preferred. In order not to limit the performance characteristics of the dressing with respect to inhibiting the activities of proteases and/or absorption and elimination of aqueous liquids, it should be guaranteed that the content of polyacrylate particles with respect to the carrier material is, in particular, not more than 80 weight % and in particular not more than 75 weight % (with respect to the carrier material of all particle-containing layers).

In accordance with a further idea, the present invention also concerns the use of a particle mixture, in particular, in a dressing comprising polyacrylate particles of different sizes for blocking or inhibiting proteases in a wound, wherein the particle mixture comprises

a) 5 to 100 weight % of particles having a particle size x with x≦300 μm, and
b) 0 to 95 weight % of particles having a particle size x with x>300 μm.

The particle mixture of this application preferentially comprises

a) 20 to 100 weight % of particles having a particle size x with x≦300 μm, and
b) 0 to 80 weight % of particles having a particle size x with x>300 μm.

The particle mixture of this application preferentially comprises

a) 20 to 98 weight % of particles having a particle size x with 45≦x≦300 μm, and
b) 2 to 80 weight % of particles having a particle size x with x>300 μm.

In a further advantageous application, the polyacrylate particles having a particle size x with x≦300 are used to produce a means for blocking or inhibiting proteases by compartmentalization, in particular, metallo proteases and preferentially matrix metallo proteases in chronic wounds. These polyacrylates may additionally advantageously be used to inhibit proteases, in particular metallo proteases and preferentially matrix metallo proteases for tissue build-up and/or for regulating the tissue build-up in chronic wounds.

Chronic wounds may be defined as wounds, the healing process of which differs from normal wound healing in one or all stages of wound healing. A chronic wound may develop from acute, normally healing wounds e.g. through wound infection, and is characterized by a prolonged healing process. A wound can thereby change from an acute to a chronic state at any stage of wound healing. Chronic wounds are clinically defined as wounds, the healing of which requires more than 6 to 8 weeks, wherein this definition is not correct for all clinical pictures. A chronic wound is rather a diagnosis which is supported on the clinical experience of the medial staff.

Chronic wounds are generated, in particular, by mechanical stress (decubitus, pressure ulcers, decubitus ulcers), venous insufficiency (ulcus cruris venosum, venous ulcers), arteriosclerotic vessel changes (ulcus cruris arteriosum, arterial ulcers), neuropathic changes (diabetic foot syndrome, neuropathic ulcers) but also as a consequence of autoimmune diseases, tumors (exulcerating tumors) or radiation injuries in tumor therapy.

A decubitus is defined as a trophic disturbance of tissue (mainly skin and subcutaneous tissue) with necrosis, mazeration, possibly infection, and is caused by external (long-term) pressure with compression of vessels and local ischemia. Decubital ulcers develop mainly in persons who are confined to bed, in particular, at parts of the body where the skin directly contacts the bone but e.g. also under badly adjusted prostheses and excessively tight plaster bandages.

Decubitus is classified into the following stages. In particular, decubita of stage II, stage III and stage IV are known as chronic wounds:
decubitus stage I: persisting localized erythema which remains even after relieve. The erythema is exactly defined and can harden or overheat. The skin is still sound.
decubitus stage II: in this phase, blisters form and the skin is abraded with partial loss of the skin. The epidermis is damaged up to parts of the dermis. This phase is characterized by a surface wound or flat ulcer.
decubitus stage III: in this advanced state, the loss of all skin layers can be observed. Moreover, the subcutaneous tissue is damaged and necroses may be observed which may extend to the muscle tissue below. Experience has shown that the necrotic tissue must be confined in order to be able to asses the overall extent of the tissue damage. Decubitus III clinically shows in the form of an open deep ulcer.
decubitus stage IV: This extremely critical stage is characterized by the loss of all skin layers with extensive destruction, tissue necrosis or damage of muscles, bones or supporting structures (tendons, joint capsules). Decubitus IV clinically shows in the form of a large-surface open and deep ulcer.

For this reason, in a further development, the present invention also concerns the use of a particle mixture of polyacrylate particles having each of the above-mentioned compositions, as a means for inhibiting proteases, in particular metallo proteases for the treatment of decubitus, ulcus cruris venosum, ulcus cruris arteriosum or diabetic foot syndrome and for the production of a means for the treatment of these diseases. The present invention concerns, in particular, also the use of a particle mixture of polyacrylate particles comprising

a) 20 to 100 weight % of particles having a particle size x with x≦300 μm and
b) 0 to 80 weight % of particles having a particle size x with x>300 μm as means for inhibiting proteases, in particular metallo proteases, for the treatment of decubitus, ulcus cruris venosum, ulcus cruris arteriosum or diabetic foot syndrome and for the production of a means for the treatment of these diseases. The tissue build-up is thereby supported by eliminating the mechanisms which disturb wound healing, which clinically shows in an improvement of the wound state and reduction of the wound size.

In one further advantageous application, the polyacrylate particles are used for inhibiting proteases by compartmentation, in particular metallo proteases, and for producing a means for inhibiting proteases by compartmentation, in particular metallo proteases, for decubitus, ulcus cruris venosum, ulcus cruris arteriosum or diabetic foot syndrome. These polyacrylate particles may also advantageously be used for inhibiting metallo proteases or matrix metallo proteases for tissue build-up and/or for regulating the tissue build-up for these diseases.

At this point, it must be emphasized that the above-mentioned features of alternative or preferred embodiments of the invention are not to be limited to the individual alternatives or preferred embodiments. In connection with the present invention, rather a combination of embodiments or a combination of each individual feature of an alternative form with features of another alternative embodiment is also to be regarded as subject matter in accordance with the invention. The invention is also not to be understood as being confined to the following description of the drawing.

The invention is explained below with reference to the drawings.

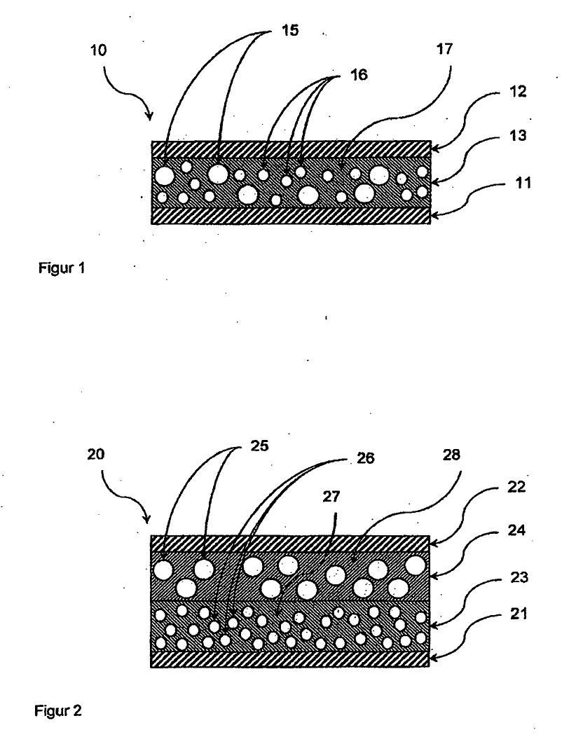

FIGS. 1 to 4 show cross-sectional views of four different inventive dressings with polyacrylate particles;

FIG. 5 shows different binding of metallo proteases from the exudate of chronic wounds in dependence on the particle size of the polyacrylate particles.

FIG. 1 shows a first embodiment of an inventive dressing (10). The dressing has a first separating layer (11) to be applied to a wound in accordance with the intended use, and a first cover layer (12). A further layer (13) is located between these two layers, which comprises a carrier material (17) and a particle mixture of polyacrylate particles (15, 16). The particle mixture contains identical portions of polyacrylate particles (16) of a first particle size x with 45≦x≦300 μm and polyacrylate particles (15) of a second particle size x with 600≦x≦850 μm. The particle mixture therefore contains 50 weight % of polyacrylate particles (16) of a first particle fraction with a first particle size x with 45≦x≦300 μm and 50 weight % of polyacrylate particles (15) of a second particle fraction with a second particle size x with 300<x≦850 μm (with respect to the overall weight of the particles). The carrier material (17) for the particle mixture is a fiber material of hydrophilic staple fibers comprising 94 weight % of hydrophilic cellulose fibers and 6 weight % of polypropylene fibers. The carrier material (17) and the polyacrylate particles (15, 16) are processed into a layer in an air-laid process, wherein the layer has a weight per unit area of 360 g/m². The portion of fiber material in the layer amounts to 198 g/m², wherein the portion of polyacrylate particles (15, 16) is 162 g/m². The portion of polyacrylate particles (15, 16) therefore corresponds to 45 weight % with respect to the carrier material (17). The first separating layer (11) and the first cover layer (13) are produced from a nonwoven of a hydrophobic fiber material of polypropylene fibers. This dressing therefore has a first separating layer as a wound contact layer which does not stick to the wound.

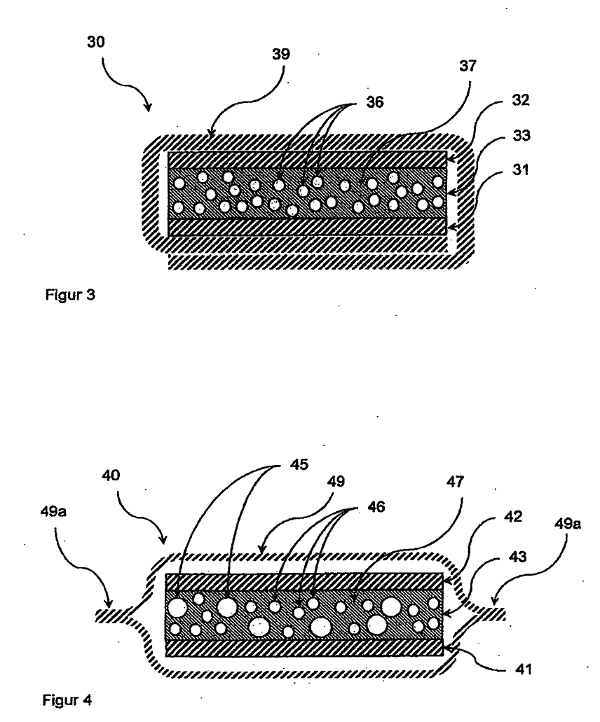

FIG. 2 shows an alternative dressing (20). This dressing has a wound contact layer (21) of a hydrophobic nonwoven of polyester fibers. A first layer (23) containing polyacrylate particles, a second layer (24) containing polyacrylate particles, and a cover layer (22) are disposed on top of this wound contact layer (21). The polyacrylate particles (26) of the first particle-containing layer (23) thereby have a particle size x with 150≦x≦300 μm and the polyacrylate particles (25) of the second particle-containing layer (24) have a particle size x with 600≦x≦1500 μm. The first particle-containing layer (23) contains 126 g/m² of polyacrylate particles having a particle size x with 150≦x≦300 μm and 198 g/m² of hydrophilic staple fibers. The second particle-containing layer (24) contains 42 g/m² of polyacrylate particles having a particle size x with 600≦x≦1500 μm (25) and 63 g/m² of hydrophilic staple fibers. The first layer (23) therefore has a weight per unit area of 315 g/m² and the second layer (24) has a weight per unit area of 104 g/m², wherein the particle portion with respect to the carrier material of the particle-containing layers (23, 24) is 40 weight % and wherein 75 weight % of polyacrylate particles with a particle size x with 150≦x≦300 μm (with respect to the overall amount of polyacrylate particles) are contained in the first layer (23), and 25 weight % of polyacrylate particles with a particle size x with 600≦x≦1500 μm (with respect to the overall amount of polyacrylate particles) are contained in the second layer (24). The carrier material (27, 28) of the first and the second particle-containing layer (23, 24) is a hydrophilic fiber material (hydrophilic staple fibers) of 90% cellulose fibers and 10 weight % of polypropylene fibers, wherein the first and also the second particle-containing layer are produced in an air-laid process. In the present case, the two layers were produced in successive steps in an in-line process. The overall weight of the two particle-containing layers (23, 24) is 420 g/m². The cover layer (22) consists of a water vapor-permeable polyurethane film having a thickness of 60 μm.

FIG. 3 shows a third alternative dressing (30). This dressing consists of a particle-containing layer (33) which is covered on both sides by a tissue (31, 32) of cellulose fibers. The weight per unit area of the tissue layers is 18 g/m². This layer composite is surrounded by a cover (39) of a nonwoven (staple fiber nonwoven of 40% polyamide fibers and 60% viscose fibers) which is permeable for the wound exudate, wherein the cover surrounds the layer composite in such a fashion that it is formed having two layers on the side facing away from the wound in the ready-to use state of the dressing. These two layers are held together by hot-melt application (not shown). The particle-containing layer consists of 280 g/m² of polyacrylate particles (Favor SMX 9170-Stockhausen, Krefeld) and 128 g/m² fluff (wood pulp fibers of a fiber length of <5 mm. The polyacrylate particles (36) have a particle size x with 150≦x≦300 μm, wherein these particles were sieved out from the raw product (Favor SMX 9170). These particles consist of a linked sodium polyacrylate and have a water content of maximally 4 weight % of water with respect to the polyacrylate particles. The dressing therefore comprises 100 weight % of polyacrylate particles having a particle size x with 150≦x≦300 μm (with respect to the overall content of particles) and 60 weight % of polyacrylate particles with respect to the carrier material.

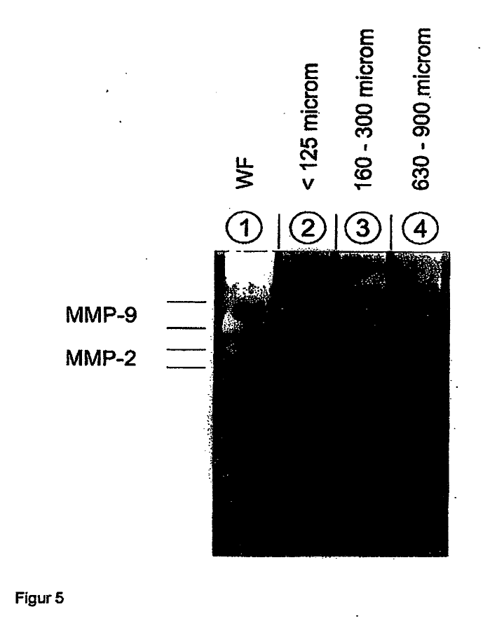

FIG. 4 shows a fourth alternative dressing (40). This dressing has a similar construction to the dressing shown in FIG. 1, wherein the dressing comprises a particle-containing layer (43) which is covered on both sides by a tissue (31, 32). The tissue layers consist of 100% cellulose fibers and have a weight per unit area of 18 g/m². The particle-containing layer (43) consists of a carrier material (47) and a particle mixture of polyacrylate particles (45, 46). The particle mixture contains 65 g/m² of polyacrylate particles (46) of a first particle size x with 150≦x≦300 μm and 97 g/m² of polyacrylate particles (45) of a second particle size x with 600≦x≦850 μm. The particle mixture therefore contains 40 weight % of polyacrylate particles (46) of a first particle fraction with a first particle size x with 150≦x≦300 μm and 60 weight % of polyacrylate particles (45) of a second particle fraction having a second particle size x with 600<x≦850 μm (with respect to the overall weight of particles). The individual particle fractions were sieved out from polyacrylate particles (Favor PAC 300-Stockhausen, Krefeld). These particles consist of a linked sodium polyacrylate and have a water content of 3.0 weight % of water with respect to the polyacrylate particles. The carrier material (47) for the particle mixture is a fiber material of hydrophilic staple fibers having 94 weight % of hydrophilic cellulose fibers and 6 weight % of polypropylene fibers. The carrier material (47) and the polyacrylate particles (45, 46) are processed into a layer in an air-laid process, wherein the layer has a weight per unit area of 360 g/m². The portion of fiber material in the layer is 198 g/m², wherein the portion of polyacrylate particles (45, 46) is 162 g/m². The portion of polyacrylate particles (45, 46) therefore corresponds to 45 weight % with respect to the carrier material (47). The dressing is furthermore surrounded by an outer cover (39) of a hydrophobic knitted fabric of polyethylene, which has a thickness of 0.8 mm in the relieved state. This knitted fabric prevents sticking to the wound and has a good modelling capacity such that the overall dressing can be adjusted to the wound base. The cover is thermo-sealed on the circumferential edges (39a, 39b) on all sides. FIG. 5 shows different types of bonding of metallo proteases on polyacrylate particles.

In the following example, polyacrylate particles (linked polyacrylate, Favor PAC 300, company Stockhausen, Krefeld, Germany) were divided into different size ranges by sieving. Fractions having particle sizes <125 μm, between 160 and 300 μm, and between 630 and 900 μm were examined. The particles were pre-activated with Ringer solution (8.6 g NaCl, 0.3 g KCl, 0.3 g CaCl₂.2H₂O ad 1000 ml water). 0.2 g polyacrylate particles and 36.94 ml Ringer solution were thereby left for 24 hours in a closed container and any excess Ringer solution was discarded. 100 mg of the pre-activated polyacrylate particles were incubated with 20 μg wound exudate protein in 50 μl for 2 hours with constant shaking at room temperature. The wound exudate protein was obtained from patients having ulcus cruris, the protein content was determined with the biorad Proteinassay (Bio-Rad DC Protein Assay Kit II, catalogue number 500-0112) and adjusted with Ringer solution to 20 μg/50 μl. The excess was removed after incubation and the treated polyacrylate superabsorber was washed 3 times with an excess (5 w/w) of washing buffer (10 g Bovine Serum Albumin (Sigma A-2153), 8.6 g NaCl, 0.3 g KCl, 0.33 g CaCl₂.2H₂O ad 1000 ml water). The polyacrylate treated in this fashion was mixed with 1 volume (v/w) 2×SDS gel sample buffer, boiled at 95° C. for 10 minutes in the water bath and subsequently stored at −20° C. or directly subjected to gelatine zymography. Towards this end, an aliquot of the wound fluid, the excess treated with polyacrylate, and the proteins bound to the polyacrylate were applied to an SDS gel containing gelatine, and were electrophoretically separated.

This technology is extremely sensitive and is based on a separation of the proteases in an SDS gel which contains protease substrate (gelatine) at the same time.

The gel is composed of (all chemicals from the company Sigma-Aldrich Chemie GmbH, 89555 Steinheim, Germany):
3.3 ml gelatine solution (gelatine, porcine skin (CAS-Nr. 9000-70-8), 3 mg/ml H₂O),
0.85 ml distilled H₂O,
2.5 ml 1.5M Tris (Tris(hydroxymethyl)aminomethane/HCl/0.4% SDS solution (sodiumdodecylsulfate solution), pH 8.8,
3.35 ml 30% acrylamide/0.8% bisacrylamide solution,
50 μl (10%, w/v) ammonium persulfate and
5 μl TEMED (N,N,N′,N′-tetramethylene diamine); the upper gel corresponds to
3.075 ml distilled H₂O,
0.625 ml 0.5 M Tris (Tris(hydroxymethyl)aminomethane)/HCl/0.4% SDS solution (sodiumdodecylsulfate solution), pH 6.8,
0.75 ml 30% acrylamide/0.8% bisacrylamide solution,
50 μl (10%, w/v) ammonium persulfate and
5 μl TEMED (N,N,N′,N′-tetramethylene diamine)

The gels may be used as so-called minigels or in other apparatus as conventional zymograms for separating the protein mixture. The expert is familiar with apparatus and methods of this type.

The sample application buffer may be any protein application buffer which must not contain any reducing substances. After separation, the proteases are renaturated (washing for 2×15 minutes in 2.5% Triton-X-100 in H₂O, then washing for 2×15 minutes in 50 mM Tris/HCl pH 7.4; 5 mM CaCl₂) and incubated in the last washing buffer for 24-48 hours. The renaturated metallo proteases decompose the gelatine substrate in their direct vicinity. When these zymograms are died with a protein colorant (Coomassie CAS-Nr. 6104-59-2, company Sigma-Aldrich Chemie GmbH, 89555 Steinheim, Germany), then decolorized according to standard protocols, the non-decomposed gelatine then appears homogeneously blue in the gel (in the illustration in FIG. 5 shown in grey/black). Only at locations of proteolytical activity where the gelatine was decomposed, there are imposing clear transparent bands, so-called “eat-through bands” (shown in FIG. 5 in white on grey/black background). The type of enzyme could be determined on the basis of the typical pattern and the molecular weight range (not shown herein) determined by a protein marker. The above-described method and variations thereof (e.g. Herron et al., J Biol. Chem. 1986 261:2814-8) are familiar to the expert. A semi quantitative evaluation was performed by the image analysis Software ImageJ (Image Processing and Analysis in Java, http://rsb.info.nih.gov/ij/index.html, last accessed on Nov. 19, 2007) through densitometric evaluation of the band intensities in accordance with the software instructions http://rsb.info.nih.gov/ij/docs/index.html, last accessed on Nov. 19, 2007.

These experiments showed clear bonding of the metallo proteases to the different polyacrylate particle fractions in dependence on the particle size. The semi quantitative evaluation is shown in table 2.

20 μl of the diluted initial wound exudate was applied to the first strip (WF). The bound wound exudate was released in 25 μl sample application buffer through heating in the 2., 3. and 4. strips, wherein only a very small part remains on the polyacrylate particles. The proteins obtained in this fashion were applied to the gelatine zymograms and developed. The band intensities of the first strip (wound exudate) were thereby determined as reference with 100. The intensities of the strips 2, 3 and 4 are stated in percent with respect to the reference.

TABLE 2

| | Strip 1 | Strip 2 | Strip 3 | Strip 4 |
|---|---|---|---|---|
| | WF | <125 μm | 160< × <300 μm | 630< × <900 μm |
| MMP-9 | 100% | 61.2%(±7.3%) | 79.3% | 10.9% |
| | (±0%) | | (±5.7%) | (±2.8%) |
| MMP-2 | 100% | 44.5% | 34.2% | 7.2% (±1.0%) |
| | (±0%) | (±4.7%) | (±2.0%) | |

It is clearly shown that in strip 2 (polyacrylate particles having a particle diameter of less than 125 μm) and strip 3 (polyacrylate particles having a particle diameter of 160 to 300 μm) the signals for the MMP-9 and MMP-2 bands are considerably stronger. Due to the washing steps after incubation of the polyacrylate particles with the wound exudate, strong bonding must be assumed. Polyacrylate particles having a particle diameter x with 160<x<300 bind the metallo protease MMP-9 approximately seven times better than polyacrylate particles having a particle diameter x with 630<x<900. The same particle fraction binds the metallo protease MMP-2 approximately four times better than polyacrylate particles having a particle diameter x with 630<x<900.

Clinical tests have proven the inhibition of the matrix metallo proteases as a result of which chronic wounds healed. In this test, three representative patients who suffer from an open ulcer on a lower leg (ulcus cruris) were treated with a dressing having the structure as described in FIG. 4. The particle fraction of 40 weight % of polyacrylate particles was thereby selected with a fraction of 150<x≦300 μm in order to inhibit excessive protease amounts for these patients and induce wound healing. Special attention must thereby be paid to the initiation of the granulation tissue to enable formation of new connective tissue. The dressing was changed every 24 hours or every other day. The results are combined in the following table:

TABLE 3

| | start | | | | end | | | |
|---|---|---|---|---|---|---|---|---|
| | | | granulation | wound | | | granulation | wound |
| | day | coating | tissue | size | day | coating | tissue | size |
| Patient 1 | 1 | 4 | 1 | 25 cm² | 34 | 1 | 4 | 20 cm² |
| Patient 2 | 1 | 4 | 1 | 20 cm² | 12 | 1 | 4 | 19 cm² |
| Patient 3 | 1 | 3 | 2 | 12 cm² | 21 | 0 | 5 | 5 cm² |

The wound state of the ulcers was clinically semi-quantitatively detected on a scale of 0 to 5. The wound size was determined by plane geometry, with the following meaning:

0=no coating
1=20% of the wound surface was covered with a fibrin coating
2=40% of the wound surface was covered with a fibrin coating
3=60% of the wound surface was covered with a fibrin coating
4=80% of the wound surface was covered with a fibrin coating and
5=100% of the wound surface was covered with a fibrin coating

The granulation tissue was similarly quantified:

0 no granulation tissue recognizable,
1=20% of the wound surface was characterized by impressive red granulation tissue,
2=40% of the wound surface was characterized by impressive red granulation tissue,
3=60% of the wound surface was characterized by impressive red granulation tissue,
4=80% of the wound surface was characterized by impressive red granulation tissue,
5=100% of the wound surface was characterized by impressive red granulation tissue.

The treatment was thereby successful in changing the pathological state of chronic wounds into a normal natural healing process.

1-15. (canceled)

16. A dressing for inhibiting proteases in a wound and/or for hydroactive wound treatment, the dressing comprising:
a carrier material; and
a particle mixture containing polyacrylate particles, wherein the particle mixture comprises 5 to 100 weight % of particles having a particle size x, with x≦300 μm, as a means for inhibiting proteases in the wound, and 0 to 95 weight % of particles having a particle size y, with y>300 μm, as a means for absorbing and/or eliminating aqueous solutions, the dressing having at least 10 weight % of polyacrylate particles with respect to an overall amount of carrier material.

17. The dressing of claim 16, wherein the particle mixture contains 5 to 100 weight % of particles having the particle size x, with 45<x≦300 μm, as a means for inhibiting proteases in the wound, and 0 to 95 weight % of particles having the particle size y with 300<y≦1500 μm, as a means for absorbing and/or eliminating aqueous solutions.

18. The dressing of claim 16, wherein the particle mixture contains 20 to 98 weight % of particles having the particle size x, with 150<x≦300 μm, as a means for inhibiting proteases in the wound, and 2 to 80 weight % of particles having the particle size y with 600<y≦850 μm, as a means for absorbing and/or eliminating aqueous solutions.

19. The dressing of claim 16, wherein the particle mixture contains 50 to 100 weight % of particles having the particle size x, with x≦300 μm, as a means for inhibiting proteases in the wound, and 0 to 50 weight % of particles having the particle size y with y>300 μm, as a means for absorbing and/or eliminating aqueous solutions.

20. The dressing of claim 16, wherein the particle mixture contains 5 to 50 weight % of particles having the particle size x, with x≦300 μm, as a means for inhibiting proteases in the wound, and 50 to 95 weight % of particles having the particle size y with y>300 μm, as a means for absorbing and/or eliminating aqueous solutions.

21. The dressing of claim 16, wherein the polyacrylate particles comprise at most 10 weight % of water with respect to an overall weight of the polyacrylate particles.

22. The dressing of claim 16, wherein the polyacrylate particles have a linked and/or crosslinked and/or surface-linked polyacrylate.

23. The dressing of claim 16, wherein the dressing is a multilayer dressing.

24. The dressing of claim 16, further comprising a cover which surrounds the particle mixture and the carrier material for the particle mixture.

25. The dressing of claim 16, further comprising a wound contact layer which separates the particle mixture and the carrier material from a wound.

26. The dressing of claim 16, wherein the carrier material for the particles is a fiber material which contains, as first fibers, staple fibers of synthetic and/or natural polymers.

27. The dressing of claim 16, wherein the dressing comprises a first particle-containing layer and at least one second particle-containing layer, wherein the first layer comprises particles of particle size x and the second layer comprises particles of particle size y.

28. The dressing of claim 24, wherein the cover is a nonwoven, a knitted fabric, a fabric or a woven fabric.

29. The dressing of claim 16, further comprising a wound contact layer which comprises no particles.

30. Use of a particle mixture consisting of polyacrylate particles of different sizes for inhibiting proteases in a wound, wherein the particle mixture comprises 20 to 100 weight % of particles having a particle size x with x≦300 μm, and 0 to 80 weight % of particles having a particle size y with y>300 μm.

31. Use of particle mixture of claim 30, wherein the particle mixture comprises 20 to 98 weight % of particles having the particle size x with 45≦x≦300 μm, and 2 to 80 weight % of particles having the particle size y with y>300 μm.
